(19) United States
(12) Patent Application Publication
(10) Pub. No.: US 2026/0026891 A1
(43) Pub. Date: Jan. 29, 2026

(54) Extended Reality Systems and Method for Dental Surgery

(71) Applicants: Katya Archambault, La Jolla, CA (US); Thanos Kristallis, La Jolla, CA (US); 9337-026 Québec Inc. (Auger Groupe Conseil), Trois-Rivières (CA)

(72) Inventors: Marcel Lafontaine, Victoriaville (CA); Jonathan Marcoux, Trois-Rivières (CA); Alan Marchand, Trois-Rivières (CA); Katya Archambault, La Jolla, CA (US); Thanos Kristallis, La Jolla, CA (US)

(21) Appl. No.: 19/277,953

(22) Filed: Jul. 23, 2025

Related U.S. Application Data
(60) Provisional application No. 63/674,530, filed on Jul. 23, 2024.

(51) Int. Cl. A61B 34/20

(52) U.S. Cl. CPC A61B 34/20; A61B 2034/2065

(57) ABSTRACT

A method for superimposing visual data relative to a patient using a mixed reality device is provided. The method includes: accessing a plurality of 3D scans of the patient's face; extracting patient-specific facial features from the plurality of 3D scans; fine-tuning a pre-trained generic 3D facial recognition AI model using the patient-specific facial features to more accurately recognize the patient's face; performing real-time recognition and tracking of the patient's face on the mixed reality device using the fine-tuned model; and superimposing the visual data relative to the patient's tracked face using the mixed reality device. A corresponding system is also provided.

### RELATED APPLICATION

This application claims the benefit of and priority to U.S. provisional patent application No. 63/674,530 filed on Jul. 23, 2024, and titled “EXTENDED REALITY SYSTEMS AND METHOD FOR DENTAL SURGERY”, the contents of which are hereby incorporated by reference in their entirety.

### TECHNICAL FIELD

The technical field generally relates to extended reality (XR), and more specifically to systems and method for enhancing dental procedures using virtual reality (VR), augmented reality (AR), and/or mixed reality (MR) devices.

### BACKGROUND

Digital dental procedures have transformed dentistry by allowing dentists to design and plan treatments using digital data from various sources like Ceph, MRI, FaceCapture, or ModJaw. This shift towards digitization brings many advantages, such as enhanced precision, shorter chair time, and better patient outcomes. However, during the actual execution of these procedures in a dental chair, dentists still rely on traditional methods with limited real-time visualization support.

During a dental procedure, dentists must position dental implants, drill, or place instruments within the oral cavity while maintaining accurate alignment and avoiding vital structures like nerves. The current lack of direct visualization in these situations can result in errors, longer chair time, and potential discomfort for patients.

Therefore, there remains a need for systems and method that can provide improved real-time visualizations during dental procedures.

### SUMMARY

In an embodiment, a method for superimposing visual data relative to a patient using a mixed reality device is provided. The method including: accessing a plurality of 3D scans of the patient's face; extracting patient-specific facial features from the plurality of 3D scans; fine-tuning a pre-trained generic 3D facial recognition AI model using the patient-specific facial features to more accurately recognize the patient's face; performing real-time recognition and tracking of the patient's face on the mixed reality device using the fine-tuned model; and superimposing the visual data relative to the patient's tracked face using the mixed reality device.

In an embodiment, a computer-implemented method for training a personalized artificial intelligence (AI) model for real-time recognition and tracking of a patient's face is provided. The method includes: accessing three-dimensional (3D) features of the patient's face; partitioning the 3D features into a training set of 3D features and a testing set of 3D features; accessing a pre-trained generic AI model of a generic face, the generic AI model being configured to generate visual data about the generic face based on 3D features thereof; fine-tuning the pre-trained generic AI model based on the 3D features of the training set; and evaluating a performance of the fine-tuned AI model based on the testing set.

In an embodiment, a system for superimposing visual data relative to a patient is provided. The system includes: a controller; a memory communicably connected to the controller and storing a plurality of executable instructions; an imaging device communicably connected to the controller and configured to capture 3D scans of a patient's face; and a mixed reality device communicably connected to the controller, wherein: the controller is configured to cause the system to, upon executing the plurality of executable instructions: extract patient-specific facial features from the plurality of 3D scans; fine-tune a pre-trained generic 3D facial recognition AI model using the patient-specific facial features to generate visual data relative to the patient's face; perform real-time recognition and tracking of the patient's face on the mixed reality device using the fine-tuned AI model; and superimpose the visual data on the patient's tracked face using the mixed reality device.

### BRIEF DESCRIPTION OF THE DRAWINGS

The features and advantages of the present disclosure will become apparent from the following detailed description, taken in combination with the appended drawings, in which:

FIG. 1 is a schematic representation of a system for superimposing visual data relative to a patient in accordance with some implementations of the present technology;

FIG. 2 is a schematic representation of a pipeline for generating the visual data in accordance with some implementations of the present technology;

FIG. 3 is a flow diagram showing operations of a method for superimposing visual data relative to a patient using a mixed reality device in accordance with some embodiments of the present technology; and

FIG. 4 is a schematic representation of a Mixed-Reality device in accordance with some embodiments of the present technology.

It is to be understood that throughout the appended drawings and corresponding descriptions, like features are identified by like reference characters. Furthermore, it is also to be understood that the drawings and ensuing descriptions are intended for illustrative purposes only and that such disclosures are not intended to limit the scope of the claims.

### DETAILED DESCRIPTION

In the following description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The implementations, geometrical configurations, materials mentioned and/or dimensions shown in the figures are optional, and are given for exemplification purposes only.

Moreover, it will be appreciated that positional descriptions such as “above”, “below”, “forward”, “rearward”, “left”, “right” and the like should, unless otherwise indicated, be taken in the context of the figures only and should not be considered limiting. Moreover, the figures are meant to be illustrative of certain characteristics of the track status-monitoring system and of the vehicle comprising the same and are not necessarily to scale.

To provide a more concise description, some of the quantitative expressions given herein may be qualified with the term “about”. It is understood that whether the term “about” is used explicitly or not, every quantity given herein is meant to refer to an actual given value, and it is also meant to refer to the approximation to such given value that would reasonably be inferred based on the ordinary skill in the art, including approximations due to the experimental and/or measurement conditions for such given value.

In the following description, an implementation is an example or implementation. The various appearances of “one implementation”, “an implementation” or “some implementations” do not necessarily all refer to the same implementations. Although various features may be described in the context of a single implementation, the features may also be provided separately or in any suitable combination. Conversely, although the invention may be described herein in the context of separate implementations for clarity, it may also be implemented in a single implementation. Reference in the specification to “some implementations”, “an implementation”, “one implementation” or “other implementations” means that a particular feature, structure, or characteristic described in connection with the implementations is included in at least some implementations, but not necessarily all implementations.

It is to be understood that the phraseology and terminology employed herein is not to be construed as limiting and are for descriptive purpose only. The principles and uses of the teachings of the present disclosure may be better understood with reference to the accompanying description, figures and examples. It is to be understood that the details set forth herein do not construe a limitation to an application of the disclosure.

Furthermore, it is to be understood that the disclosure can be carried out or practiced in various ways and that the disclosure can be implemented in implementations other than the ones outlined in the description above. It is to be understood that the terms “including”, “comprising”, and grammatical variants thereof do not preclude the addition of one or more components, features, steps, or integers or groups thereof and that the terms are to be construed as specifying components, features, steps or integers. If the specification or claims refer to “an additional” element, that does not preclude there being more than one of the additional element. It is to be understood that where the claims or specification refer to “a” or “an” element, such reference does not mean that there is only one of that element. It is to be understood that where the specification states that a component, feature, structure, or characteristic “may”, “might”, “can” or “could” be included, that particular component, feature, structure, or characteristic is not required to be included.

The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. It will be appreciated that the methods described herein may be performed in the described order, or in any suitable order.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The functions of the various elements shown in the figures, including any functional element labeled as a “processor”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some implementations of the present technology, the processor may be a general purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process operations and/or textual description. Such modules may be executed by hardware that is expressly or implicitly shown. Moreover, it should be understood that module may include for example, but without being limitative, computer program logic, computer program instructions, software, stack, firmware, hardware circuitry or a combination thereof which provides the required capabilities.

Software modules, or simply modules or units which are implied to be software, may be represented herein as any combination of flowchart elements or other elements indicating performance of process operations and/or textual description. Such modules may be executed by hardware that is expressly or implicitly shown, the hardware being adapted to (made to, designed to, or configured to) execute the modules. Moreover, it should be understood that module may include for example, but without being limitative, computer program logic, computer program instructions, software, stack, firmware, hardware circuitry or a combination thereof which provides the required capabilities.

With these fundamentals in place, we will now consider some examples to illustrate various implementations of aspects of the present technology.

The technology presented herein relates to systems and method for superimposing visual data relative to a patient using a mixed reality device. Broadly described, the system includes the fine-tuning of a generic artificial intelligence (AI) facial recognition model to facilitate tracking of patient anatomical structures by a mixed reality system. The mixed reality system is designed to overlay visual data relative to tracked anatomical structures during a medical procedure (e.g. dental procedure). The disclosed system and methods may be used to improve a precision and an efficiency of treatments while ensuring optimal patient comfort. As will be described in greater detail herein after, the system may provide real-time visualization of visual data relative to the patient by, for example, displaying accurate three-dimensional (3D) models of dental implants, treatment plans, restorative and surgical procedures, and other relevant information directly onto the patient's anatomy in real-time. The system may also minimize errors by providing an operator with precise guidance for instrument placement and alignment during procedures.

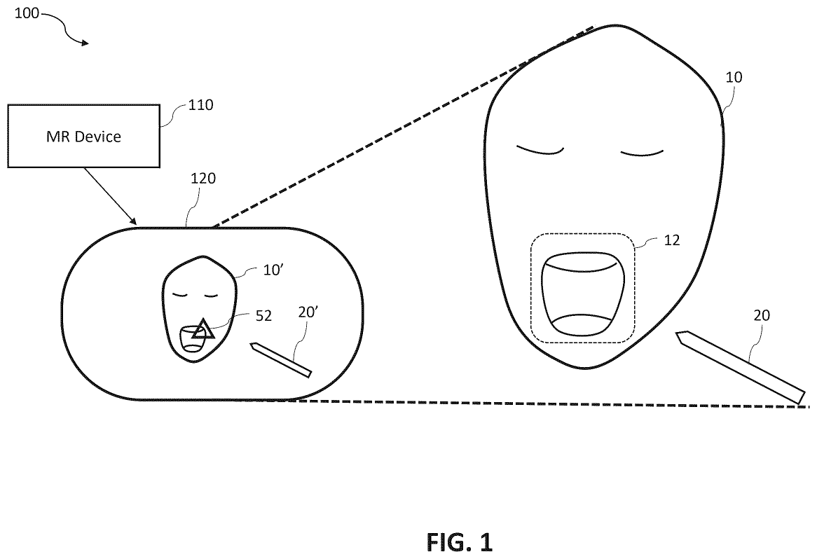

With reference to FIG. 1, an exemplary system 100 for superimposing visual data relative to a patient using a mixed reality device is shown. In the illustrated example, a dental procedure is being performed in the mouth cavity 12 of the patient's face 10. The dental procedure is performed with the help of one or more tools 20 operated by an operator, such as a dentist or surgeon. The one or more tools 20 can, for example, correspond to one or more dental instruments such as an applicator, drill, or other suitable instrument depending on the dental procedure being performed. The one or more tools 20 may be provided with markers or sensors to enable real-time detection of their position and orientation in 3D space. Although a dental procedure and corresponding tools are shown, it should be understood that this is for illustrative purposes only, and that the technology described herein can apply to other types of procedures such as, for example and without limitation, surgical procedures on other areas of a patient's body, other medical procedures, or manufacturing procedures.

In the illustrated implementation, the system 100 includes a mixed reality (MR) device 110 configured to virtually augment a physical space using digital content and allow an operator to interact with the digital content. In some implementations, the MR device 110 may include wearable device, such as a headset. It is appreciated, however, that other MR devices are possible, such as handheld devices, holographic projection devices, among others. Moreover, although an MR device will be described, it will be appreciated that other devices capable of augmenting a physical space with digital content are also possible, such as an augmented reality (AR) device and/or a virtual reality (VR) device. Said devices may be, for example, implemented as overhead devices. Finally, although various functionalities will be described hereinafter in connection with a single MR device, it is appreciated that this is for illustrative purposes only, and that functionalities of the MR device can be implemented on more than one MR device and/or with the help of hardware that is external to the MR device.

In the illustrated implementation, the MR device 110 may be worn by an operator and be configured to present stereoscopic images to the operator. It is appreciated, however, that other types of displays are possible. The MR device 110 can include any suitable user input devices. Such input devices can, for example, include one or more sensors for tracking the operator's body movements (including head movements and/or hand gestures) through preferably six degrees of freedom, one or more sensors for tracking the operator's position in the physical space, one or more sensors for capturing the operator's gaze, one or more sensors for capturing the operator's voice, one or more handheld controllers, etc. As can be appreciated, some of these sensors can be integrated in the MR device 110 while others can be separate hardware devices, such as smart cameras positioned in the physical space.

The MR device can further include sensors for detecting, mapping, and/or tracking physical elements positioned and/or moving about in physical space, such as objects (e.g., tools 20) and people (e.g., the patient). The MR device can comprise any suitable sensors capable of detecting and/or tracking objects, such as one or more cameras, depth sensors, or other scanning devices. In some embodiments, the MR device can comprise shared sensors, for example utilizing one or more integrated cameras for both detecting the operator's inputs/gestures and detecting/tracking physical objects in the physical space. In some embodiments, the MR device can cooperate with external sensors, such as a series of fixed cameras positioned throughout the physical space. External sensors can be provided in areas that require more precise detecting and/or tracking. For example, one or more dedicated cameras can be provided to closely follow the patient's face 10.

The MR device 110 augments the physical space by conveying an augmented view 120 to the operator that includes digital content presented therein. In the illustrated implementation, the augmented view 120 includes digital content in the form of visual data 52 projected in the physical space. Although visual data 52 is shown, it is appreciated that other digital content can be projected in the physical space, such as spatial audio.

While using the MR device 110, the augmented view 120 presented to the operator allows the operator to see the patient's face 10 (illustrated as a face representation 10′ in the augmented view 120), the tools 20 (illustrated as a tool representation 20′ in the augmented view 120), in addition to any environmental elements in the physical space, while seeing the visual data 52 positioned relative thereto. In the present-embodiment, the MR device 110 conveys the augmented view 120 via a projected visualization in that the visual data 52 is projected into the operator's field of view of the physical space. In other words, the face representation 10′ and tool representation 20′ correspond to a direct view of the face 10 and tool 20, for example through a display comprising a transparent or semitransparent lens. It is appreciated, however, that the augmented view 120 can be conveyed in different ways, for example via a pass-through visualization in which the operator is presented with a pass-through video stream of the physical space with the visual data 52 rendered therein. In such implementations, the face representation 10′ and tool representation 20′ can comprise digitized renderings of the face 10 and tools 20.

In some implementations, the visual data 52 may include at least one virtual object. In the context of the present disclosure, a virtual object may correspond to any 2D or 3D model that can be virtually projected in 3D within the physical space and/or relative to physical elements contained thereon, e.g. on the face representation 10′. By way of example, the virtual object can include 3D models of dental implants or treatment plans, such as computer-aided design (CAD) models for smile designs, virtual smile designs or any general virtual surgical or restorative planning that can be projected on the face representation 10′. As can be appreciated, the virtual object can include other 3D or 2D models that can enhance and/or assist the operator in performing a dental procedure or operation. For example, the virtual object can comprise a virtual screen or display configured to present information such as an image, a video, a document (such as a pdf), a webpage, or other graphical user interface for software, etc.

In some implementations, the virtual object may be freely manipulated and positioned in 3D space. For example, a virtual object comprising a virtual screen or display can be positioned by the operator such that it remains at a fixed position within the augmented view 120 of the physical space (e.g., such that it remains anchored within the physical space), or that is remains at a fixed position within the operator's field of view (e.g., in a “head-locked” position). In some implementations, a virtual object can be attached or aligned relative to physical objects in the physical space such that they follow movement thereof through 3D space. By way of example, in the illustrated embodiment, the visual data 52 comprises a virtual object in the form of a virtual dental implant that is aligned relative to the patient's face 10. More specifically, the virtual dental implant can be aligned such that in the augmented view 120, it is projected within the patient's oral cavity 12 at a position where a physical dental implant is to be installed to guide the operator in installing said implant. The virtual dental implant can be projected at the appropriate location within the patient's oral cavity 12, even as the patient moves their head and/or opens and closes their mouth.

As can be appreciated, in order to maintain alignment of a virtual object with a physical object, the MR device 110 can be configured to track a position and orientation of the physical object and update a position an orientation of the virtual object in real time in the augmented view 120. For example, this can involve utilizing one or more sensors in the MR device 110 to continuously determine a position and/or orientation of a physical object in the physical space, continuously determine a position and/or orientation of a virtual object relative to the physical object, and continuously updating a position and/or orientation of the virtual object to maintain alignment with the physical object.

Continuously determining the position and/or orientation of a physical object can be carried out using different techniques. For example, the MR device 110 can be configured to utilize sensors, such as optical sensors and depth sensors, to generate and maintain a 3D model of the physical space, including physical objects contained therein. The 3D model can be analyzed to identify objects contained therein, and to track movement of said objects as the 3D model is updated. In some implementation, the tracking of physical objects by the MR device 110 can be facilitated through the use of passive or active tags. For example, a plurality of tags with different identifiable patterns can be affixed to predetermined positions on a tool 20, and the plurality of tags can be detected and located by the sensors in the MR device 110 to infer the location of the tool 20. In some implementations, the tracking of physical objects by the MR device 110 can be facilitated through advanced object recognition. For example, in the present implementation, and as will be described in more detail hereinafter, AI-assisted facial recognition can be carried out to identify and track the patient's face 10.

Continuously determining the position and/or orientation of a virtual object relative to the physical object can also be carried out using different techniques. For example, the position and orientation of virtual object can be defined relative to a frame of reference, and a corresponding frame of reference can be identified in the physical object and aligned with the virtual object's frame of reference. In the present embodiment, the visual data 52 comprises a virtual dental implant whose position and orientation are defined relative to a model of the patient's jaw structure. The position and orientation of the patient's actual jaw structure in the physical space can be identified by the MR device 110 to align the virtual dental implant therewith in the augmented view 120. As can be appreciated, the MR device 110 can be configured to infer the position and orientation of the patient's actual jaw structure since the jaw structure may not be directly detectable or trackable by the MR device 110. For example, pre-operative scans can be carried out to model the patient's jaw structure in relation to the patient's face 10. This model can then be used by the MR device 110 to infer the position and orientation of the patient's actual jaw structure in relation to the tracking of the patient's face 10. As can be appreciated, similar techniques can be carried out to align virtual objects to other anatomical structures of the patient, including other structures in the patient's face.

In more detail now, the AI-assisted facial recognition can be carried out using a 3D facial recognition AI model that is fined-tuned using patient-specific data for more accurately tracking the patient's face.

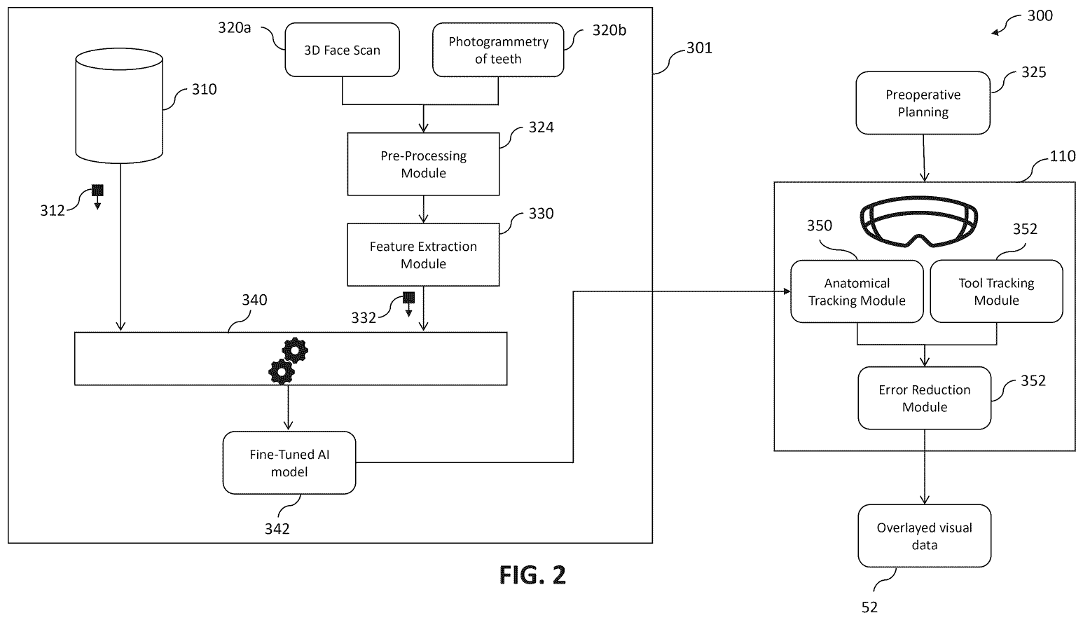

An exemplary pipeline 300 for generating a fined-tuned AI model and using the same to facilitate projecting visual data is illustrated in FIG. 2. In the illustrated implementation, an AI model generating system 301, such as a server or other computing system, accesses a plurality of 3D scans of the patient's face 10. In the context of the present disclosure, a 3D scan of the patient's face 10 is a digital representation of the patient's facial features, such as a general 3D face scan 320a, or photogrammetry of the patient's teeth 320b. The 3D scans can, for example, be represented as a 3D point cloud or 3D mesh. A 3D scan may include detailed surface data from multiple angles to create a high-resolution, three-dimensional representation of the patient's face 10. In the present implementation, the 3D scans accessed by the system 301 include 3D face scans 320a of the patient from different angles and expressions, in addition to photogrammetry of the patient's teeth 320b. The 3D scan may include intricate details such as skin texture, contours, expressions, teeth configuration and structure, among others. In some implementations, the 3D scan can be generated using equipment such as laser scanners, structured light scanners, or other photogrammetry techniques during a preoperative stage.

The system 301 executes a feature extraction module 330 using the 3D scans 320 as an input. The feature extraction module 330 may identify and isolate attributes or characteristics of the patient's face 10 based on the 3D scans 320. For example, the feature extraction module 330 may be configured to detect key points on a surface, such as corners, edges, and textures, for characterizing a geometry of the patient's face 10. The feature extraction module 330 may also generate descriptors for identified features, that may be used as compact representations for further matching and comparison. In some implementations, the feature extraction module 330 employs an Iterative Closest Point (ICP) algorithm to align multiple 3D scans to form a coherent model by minimizing differences between point clouds.

For example, the feature extraction module 330 may be configured to detect facial landmarks as key points (e.g., corners of the eyes, edges of the lips, the tip of the nose, and the jawline corners) and/or contour points that may be defined along the edges of the face and defining the general contour of the patient's face 10. It should be noted that facial scans focus on landmarks like eyes, nose, mouth, and jawline while dental scans focus on specific points on the teeth and gums, such as cusps, ridges, and occlusal surfaces.

The feature extraction module 330 may also be configured to detect geometric features such as corners of the patient's face 10 where the surface curves change sharply (e.g., corners of the eyes or mouth), edges of the patient's face 10 defined by abrupt changes in surface direction (e.g., along the nasolabial folds), or planes of the patient's face 10 defined as flat regions (e.g., the forehead or cheeks).

The feature extraction module 330 may also be configured to detect textural features of the skin texture, defined as fine variations in skin texture, including wrinkles, pores, and other surface details. It should be noted that facial scans analyze skin textures, including wrinkles and pores while dental scans focus on enamel texture, cracks, and wear patterns on the tooth surface.

The feature extraction module 330 may also be configured to detect shape descriptors such as histograms of normal to and surface of the patient's face 10 (e.g., a distribution of surface normal angles), local surface curvatures, and moment-based descriptors such as surface moments that map the overall shape of the patient's face 10.

The feature extraction module 330 may also be configured to detect local features such as details specific to small regions of the face (e.g., features around the eyes or mouth) and global features such as properties that describe the face 10 as a whole (e.g., overall proportions and symmetry).

Broadly speaking, the feature extraction module 330 is configured to take into account that the patients' face 10 has a more complex and larger-scale geometry with smooth transitions between features, while teeth have finer and more intricate details with sharper transitions and higher frequency features. Furthermore, it should be noted that facial scans may also include information about functional aspects like facial expressions and muscle movements, while dental scans typically consider dental occlusion and dental arches.

Furthermore, the feature extraction module 330 may recognize and classify patterns within the 3D scans, enabling tasks like facial recognition and object detection. In the illustrated implementation, the feature extraction module 330 extracts patient-specific facial features 332 from the 3D scans 320.

In some implementations, the 3D scans 320 can be subject to preprocessing before being provided as input to the feature extraction module 330. For example, in the present implementation, the system 301 executes a pre-processing module 324 that is configured to pre-process the 3D scans for face tracking before features are extracted therefrom.

The pre-processing module 324 may execute noise filtering to remove noise data from the 3D scans, and/or perform surface smoothing to enhance a data quality of the 3D scans for more accurate feature extraction. Additionally or optionally, the pre-processing module 324 may convert 3D data of the 3D scans to two-dimensional (2D) representations. This process may involve employing sampling and flattening techniques to create 2D images or vectors that can be more easily processed by machine learning algorithms.

It should be noted that the pre-processing steps can vary significantly whether ta given 3D scan is a 3D face scan or a dental photogrammetry, due to the differences in the structures being analyzed. More specifically, the pre-processing of a 3D face scan may include landmark-based alignment to align and standardize the orientation of the face scans using facial landmarks, expression normalization to ensure consistency in the recognition model by adjusting for different facial expressions, and/or feature extraction to emphasize features relevant to identity, such as the shape of the eyes, nose, and mouth. The pre-processing of a photogrammetry of teeth may include occlusion handling to analyze contact points between teeth, fine detail preservation to capture subtle variations in tooth texture and shape by maintaining high-resolution details, and/or anatomical segmentation performed by isolating individual teeth or specific areas for targeted analysis.

The system 301 is further configured to access a pre-trained generic facial recognition AI model 312 as a starting point for generating the fine-tuned model. Broadly speaking, the generic facial recognition AI model 312 can correspond to any AI model that is trained to generate a vector representation of one or more facial features from a face in an input image. The AI model 312 can be pre-trained on a large and/or generic dataset, i.e. a dataset comprising a large number of images of faces of different people in different poses, across different demographics, having different characteristics, and in different environmental or lighting conditions. By way of example, the AI model 312 can correspond to the FaceNet architecture trained on the VGGFace2 dataset. It is appreciated that different architectures, and/or models trained on different datasets can be used.

In the illustrated configuration, the pre-trained model 312 is accessed from an AI model database 310. In some configurations, the AI model database 310 can store a plurality of AI models, and the system 301 can be configured to select one from the plurality of AI models for use as a starting point for generating the fined-tuned model. The plurality of AI models can include a plurality of different architectures, and/or a plurality of models trained on different datasets. For example, the plurality of AI models can comprise the same architecture trained on datasets skewed to different demographics, characteristics, and/or environmental conditions to better fit the AI model to a particular demographic, set of characteristics, and/or environmental conditions. The system 301 can be configured to select an AI model based on patient characteristics and/or environmental conditions of the dental procedure. For example, if the patient is an adult, the system 301 can be configured to select a generic AI model that is pre-trained on images of adults, as opposed to a generic AI model that is pre-trained on images of children. As another example, the patient is a male, the system 301 can be configured to select a generic AI model that is pre-trained on images of men, as opposed to a generic AI model that is pre-trained on images of women.

Broadly speaking, the pre-trained model 312 may be selected based on specialized features thereof. For example, a feature of the pre-trained model 312 may be occlusion handling, referring to models that are capable of dealing with partial occlusions, such as hands covering parts of the face or wearing masks.

Another example of a feature is ensemble learning, refereeing to the usage of an ensemble of models, each trained on different aspects or datasets, to improve overall performance and robustness. For instance, combining models trained on different demographic groups to ensure fairness and accuracy across a diverse population. Yet another example of feature is domain adaptation, used to adjust pre-trained models to new domains where the data distribution may differ from the original training data. This may be useful when the application data has specific characteristics not covered by the generic model.

The pre-trained model 312 may also be selected based on the features it is optimized to detect and/or a benchmark thereof. For example, selecting a model specialized in detecting fine details for dental scans or one optimized for tracking rapid movements for face tracking in dynamic environments. In addition, the pre-trained models of the AI model database 310 may be evaluated on a set of benchmark tasks relevant to the application and selecting the one that performs best. This may involve comparing metrics like accuracy, recall, precision, F1-score, and computational efficiency.

The system 301 further executes a fine-tuning module 340 to fine-tune the pre-trained generic facial recognition AI model 312, thereby generating a fine-tuned AI model 342. Broadly speaking, executing the fine-tuning module 340 involves adapting the pre-trained generic facial recognition AI model 312 to enhance its performance in tasks like facial recognition, expression detection, and tracking relative to the patient's face 10. The pre-trained generic facial recognition AI model 312 is refined using the patient-specific features 332. During fine-tuning, the pre-trained generic facial recognition AI model 312 may be adjusted by adjusting weights of layers thereof based on the patient-specific features 332. Fine-tuning in this manner allows the AI model to better fit to characteristics of the patient's face and thus allows to more accurate recognize and track the patient's face 10 specifically.

It should be noted that, for face tracking in an AR application, the pre-trained model 312 can be selected based on its real-time performance, ability to handle diverse lighting conditions, and accuracy in tracking facial landmarks. The pre-trained model 312 can be further fine-tuned using a dataset of faces in different lighting environments and with various expressions to ensure robust performance in dynamic and varied real-world conditions.

In more detail, fine-tuning the pre-trained generic facial recognition AI model 312 may involve further training thereof using the patient-specific features 332 while adjusting the learning rate and training for multiple epochs to optimize performance. In some implementations, this can involve partitioning the patient-specific features 332 into a training dataset and a testing dataset. The training dataset is used to further train the pre-trained generic facial recognition AI model 312, and the testing dataset is used to test performance (e.g. accuracy, precision, and recall) of the further trained AI model 312 to adjust training parameters as needed until a desired performance threshold is reached, thereby obtaining the fine-tuned AI model 342.

As an example, fine-tuning a facial recognition model for a specific patient may first include obtaining multiple 3D scans or 2D images of the patient's face under different conditions. In this implementation, these images are labeled with relevant annotations, such as facial landmarks or expressions. The fine-tuning may further include splitting the data into a training set (e.g., 80%) and a testing set (e.g., 20%) and loading a pre-trained model, such as a FaceNet model trained on the VGGFace2 dataset. A training configuration may be set, for example by setting the learning rate to 0.0001 and execute a training for 50 epochs with a batch size of 16.

For example, the training may include, from epoch no. 1 to epoch no. 10, learning patient-specific features and gradually adjusting the pre-trained weights by the facial recognition model. The training may further include, from epoch no. 11 to epoch no. 20, monitoring performance of the facial recognition model on the testing dataset and adjusting the learning rate if necessary. The training may further include, from epoch no. 21 to epoch no. 50, continuously training the facial recognition model with potential adjustments to prevent overfitting.

In this implementation, performances (e.g., accuracy and precision) of the model are evaluated on the testing dataset. Once the desired performance is achieved, the resulting facial recognition model is the fine-tuned AI model 342. Broadly speaking, fine-tuning the facial recognition model may enhance an accuracy thereof. By training on patient-specific data, the model becomes more accurate in recognizing and tracking the specific patient's face. The facial recognition model is also thus tailored to the unique features and characteristics of the patient, which enhances its performances. Finally, fine-tuning of the facial recognition model leverages the knowledge embedded in the pre-trained model, requiring less data and computational resources than training from scratch.

In some implementations, performance of the fine-tuned AI model 342 are evaluated using pre-recorded videos of a patient's face from different angles, including camera and sensor data. The pre-recorded videos may be used to analyze the system's performance in real-world scenarios.

More specifically, pre-recorded videos of the patient's face from various angles, lighting conditions, and with different expressions may be used to simulate real-world scenarios using the mixed reality (MR) device 110 or any suitable device. The pre-recorded videos may be associated with data from other sensors (e.g., depth sensors, infrared cameras) to provide a comprehensive evaluation dataset. The initial performance of the model may be assessed by running the fine-tuned AI model on the pre-recorded videos to generate predictions (e.g., facial recognition, expression detection) and comparing the model's predictions against ground truth annotations in the pre-recorded videos to calculate performance metrics such as accuracy, precision, recall, and F1-score. The performance metrics may further be analyzed to identify areas where the model underperforms, such as specific angles, lighting conditions, or expressions where recognition accuracy drops and to identify patterns in errors, such as consistent misidentification in certain poses or under specific lighting conditions.

The model may further be adjusted accordingly. As an example, the training dataset may be augmented with frames from the pre-recorded videos where the model performed poorly. This can include images with challenging angles, lighting conditions, or expressions. As another example, the model may be fine-tuned again using this augmented dataset, emphasizing the difficult scenarios to improve the model's robustness. As yet another example, the learning rate may be adjusted to ensure that the model is adapting appropriately without overfitting (e.g., a lower learning rate might be used to make finer adjustments), and/or the number of training epochs and batch size may be adjusted, as increasing epochs can allow the model more time to learn from the new data, while adjusting batch size can impact the gradient updates. As yet another example, layers in the neural network may be added or modified to better capture complex features that are causing performance issues. Regularization techniques like dropout, batch normalization, or L2 regularization may also be implemented to prevent overfitting on the augmented data. As yet another example, synthetic data that mimics the challenging conditions found in the pre-recorded videos may be generated, such as varying lighting or occlusions, and/or transformations (e.g., rotation, scaling, cropping) may be applied to the existing data to create a more diverse training set.

The model may further be iteratively tested and refined. In some implementations, after adjusting the model, its performance on the pre-recorded videos may be re-evaluated from the MR device 110 and compared to previous results to assess improvements. This process may be iterative, making incremental adjustments and re-evaluating until the desired performance is reached. Once the model performs as desired on pre-recorded videos, the model may be tested with live scenarios to ensure its performances in real-time applications are maintained. A feedback loop may be implemented where the model's live performance data is periodically reviewed and used to further refine the model if needed.

As an example, an optimization of performances of a patient's facial recognition model may first include running the patient's facial recognition model using pre-recorded videos of the patient in different lighting conditions. The performance metrics may indicate low accuracy during low-light conditions. In response, the training dataset may be augmented with additional low-light images extracted from the pre-recorded videos. The patient's facial recognition model may also be fine-tuned with this augmented dataset, with a slightly reduced learning rate to allow for more precise weight adjustments. The dropout may also be increased slightly to prevent overfitting on the augmented data. Once these adjustments made, the adjusted patient's facial recognition model may be tested again on the pre-recorded videos, showing improved accuracy in low-light conditions. Performance metrics are now closer to the desired threshold. The model may thus be deployed in a live setting to track the patient's face during a procedure.

Once the fine-tuned AI model 342 has been generated, the fine-tuned AI model 342 can be provided to the MR device 110 such that it can be used to facilitate detection and tracking of anatomical structures in the patient. In the present implementation, the fined-tuned model 342 is provided to an anatomical tracking module 350 executing on the MR device 110. The anatomical tracking module 350 is configured to localize anatomical structures through visual identification and tracking using the MR device's 110 sensors. The visual identification and tracking are facilitated by enabling accurate identification and tracking of the patient's face 10 using the fine-tuned AI model 342.

More specifically, the fine-tuned model 342 is loaded onto the MR device 110, where it is utilized by the anatomical tracking module 350, and may access data captured by sensors of the MR device 110, including for example cameras and depth sensors, configured to capture real-time data of the patient's face and surrounding anatomical structures. The fine-tuned model 342 may further processes the sensor data to accurately identify the patient's face. This involves detecting facial landmarks and features that were refined during the fine-tuning process. In use, the fine-tuned model 342 continuously tracks the position and orientation of the patient's face in real-time, ensuring that any movements are accounted for.

As an example, visual data 52 may relate to an upper jaw of the patient. The visual data 52 may be aligned assuming that the upper jaw is always at a fixed distance from the upper section of the head. In other words, the fine-tuned model 342 may use the position of the head to place visual data 52 relative of a model of the upper jaw accurately. In addition or alternatively, the visual data 52 may also relate to the lower jaw of the patient. The lower jaw's position is more variable due to its movement. The fine-tuned model 342 may track the lower section of the face to dynamically adjust the placement of the visual data 52 relating to a model of the lower jaw.

As the patient moves, the fine-tuned model 342 ensures that the overlay remains accurately positioned by continuously updating the alignment based on the real-time sensor data. The MR device 110 provides visual feedback to the surgeon, displaying the 3D model (i.e., visual data 52) overlaid on the patient's actual anatomy, enabling precise guidance during the procedure.

As can be appreciated, the anatomical tracking module 350 can be executed in parallel with other tracking system in the MR device 110. For example, in the present implementation, the MR device 110 further executes a tool tracking module 352 that is configured to identify and track tools 20. The tracking method implemented by tool tracking module 352 can be different than that of the anatomical tracking module 350. For example, as described above, the tool tracking module 352 can utilise sensors in the MR device 110 to track and/or interact with passive or active sensors provided on the tools 20.

Anatomical structures identified and tracked by the MR device 110 can be used to overlay visual data 52 relative thereto in the augmented view 120 as described above. Similarly, visual data 52 can be overlayed relative to tracked tools 20 as needed. In some implementations, the MR device 110 can implement an error-reduction algorithm to more accurately maintain alignment between the visual data and the tracked anatomical structures and/or tools and adjust the projection of the visual data as needed. For example, in the present implementation, the MR device 110 executed an error reduction module 354 configured to ensure that the alignment between the visual data (such as overlays of anatomical structures or surgical plans) and the real-world view of the patient and tools remains accurate and stable. This involves continuously correcting any discrepancies that arise due to factors such as sensor noise, patient movement, or tool displacement.

The error reduction module 354 may include sensor fusion techniques and integrate data from multiple sensors (e.g., cameras, depth sensors, IMUs) to create a more accurate and reliable representation of the tracked objects using weighted averages of sensor readings to minimize the impact of noisy or inaccurate data from any single source.

The error reduction module 354 may also include real-time data processing techniques. For examples, the error reduction module 354 may process sensor data in real-time, providing continuous updates to the positions of the anatomical structures and tools and/or may employ predictive modeling techniques to anticipate the movement of the patient's face or tools based on previous data, reducing the lag between actual movement and the visual representation.

The error reduction module 354 may also include error correction techniques, such as Kalman Filtering, by estimating the true positions of tracked objects by accounting for noise and uncertainties in the sensor data, thereby predicting a next state of the object and updates the prediction with new measurements, or Particle Filtering for more complex and non-linear movements, to maintain multiple hypotheses of object positions and refine them based on sensor data.

The error reduction module 354 may also include alignment adjustment techniques. For example, the error reduction module 354 may perform dynamic adjustment techniques by continuously adjusting the alignment of the visual data based on real-time feedback. If the anatomical structure or tool deviates from its expected position, the algorithm recalibrates the visual overlay to match the new position. As another example, the error reduction module 354 may perform error minimization techniques, by minimizing the difference between the predicted and actual positions to ensure that the overlay remains accurate and stable.

The error reduction module 354 may also include compensation-for-movement techniques. For example, the error reduction module 354 may compensate for small movements by adjusting the overlay in real-time. Larger movements might trigger a re-calibration step. The error-reduction algorithm may also ensure that the visual overlay remains aligned with the tool's actual position and orientation.

In the present implementation, the MR device 110 is configured to present visual data 52 comprising virtual objects that were generated during a preoperative phase 325. For example, the preoperative phase 325 can include gathering medical imagery of the patient, such as images obtained via Cone Beam Computed Tomography (CBCT), Multi Detector Computer Tomography (MDCT), Facial Scanning, Ultrasound, VideoFlouroscopy, Echography, Doppler, Magnetic Resonance Imaging (MRI), Intraoral Scanning (IOS), 2D cephalometric and panoramic imaging, Live 3D Jaw Movement tracking via photogrammetry and other camera capturing devices, among others, and analyzing the medical imagery using different techniques, such as cephalometric analysis, periapical analysis, and jaw movement analysis, among others, in order to establish a preoperative plan. Establishing the preoperative plan can include generating one or more 3D models, for example corresponding to one or more of the patient's anatomical structures (e.g., such as the patients jaw, facial structure, one or more of the patient's teeth, etc.), to one or more implants or devices to be installed relative to the patient's anatomical structures (e.g., such as a denture, a dental implant, a surgical guide, etc.). Such 3D models can be provided to the MR device 110 as virtual objects to be presented in the augmented view 120.

As can be appreciated, by leveraging existing pre-trained models and fine-tuning said models with 3D scans that are specific to the patient, the present technology provides a highly accurate and personalized facial recognition system that can be used during dental procedures or other medical operations. More specifically, the present technology augments visual tracking of patient anatomical structures using a highly personalized AI model optimized for the patient's unique facial characteristics, thereby providing increased precision and accuracy. This may be beneficial for reducing latency and increase a response time of display visual data overlayed relative to the patient's anatomical structure in an augmented view, thereby improving patient's safety and reducing risk of complications.

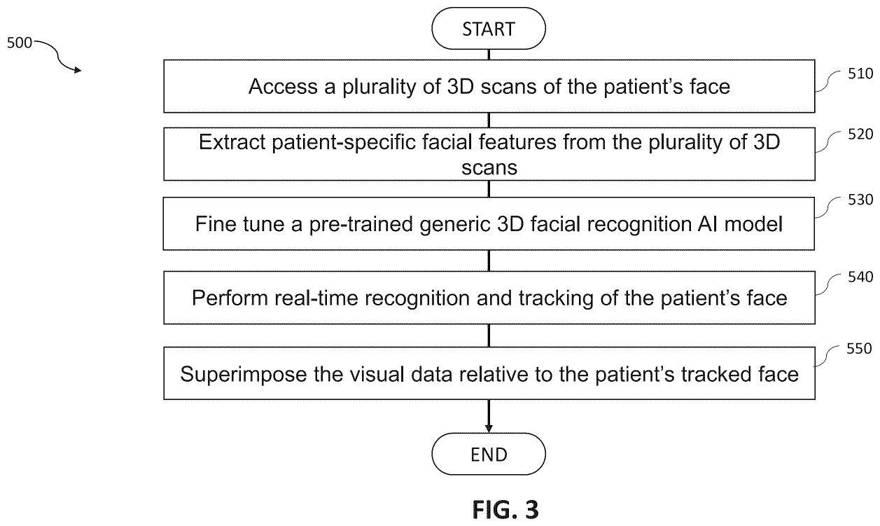

With reference now to FIG. 3, a flowchart of a method 500 for superimposing visual data relative to a patient using a mixed reality device is shown according to a possible implementation of the present technology. In one or more aspects, the method 500 or one or more steps thereof may be performed by one or more processors or computer systems, such as the AI model generating system 301 and/or the MR device 110. The method 500 or one or more steps thereof may be embodied in computer-executable instructions that are stored in a computer-readable medium, such as a non-transitory mass storage device, loaded into memory and executed by one or more processors. Some steps or portions of steps in the flowchart may be omitted or changed in order.

The method 500 includes a first operation 510 of accessing a plurality of 3D scans of the patient's face. In some implementations, the plurality of 3D scans comprises a plurality of 3D face scans of the patient from different angles and expressions.

The method 500 continues with extracting, at operation 520, patient-specific facial features from the plurality of 3D scans.

The method 500 continues with fine-tuning, at operation 530, a pre-trained generic 3D facial recognition AI model using the patient-specific facial features to generate visual data relative to the patient's face. In some implementations, the pre-trained generic 3D facial recognition AI model may be fine-tuned by adjusting weights of layers of the generic 3D facial recognition AI model based on the patient-specific facial features.

The method 500 continues with performing, at operation 540, real-time recognition and tracking of the patient's face on the mixed reality device using the fine-tuned model.

The method 500 continues with superimposing, at operation 550, the visual data relative to the patient's tracked face using the mixed reality device. In some implementations, the visual data includes a 3D model associated with a facial structure of the patient. In this implementation, the facial structure may be a dental structure of the patient. For example, the 3D model may include at least one of: a 3D model of a denture, a 3D model of a dental implant, a 3D virtual surgical guide, a 3D representation of a treatment plan, a 3D model of the patient's tooth or other facial and/or anatomical structures. A superimposition of the visual data may include determining a real-time position of patient facial structures in 3D space according to the patient's tracked face, and projecting the 3D model in alignment with the determined position of the facial structure.

It will be appreciated that at least some of the operations of the method 500 may also be performed by computer programs, which may exist in a variety of forms, both active and inactive. Such as, the computer programs may exist as software program(s) comprised of program instructions in source code, object code, executable code or other formats. Any of the above may be embodied on a computer readable medium, which include storage devices and signals, in compressed or uncompressed form. Representative computer readable storage devices include conventional computer system RAM (random access memory), ROM (read only memory), EPROM (erasable, programmable ROM), EEPROM (electrically erasable, programmable ROM), and magnetic or optical disks or tapes. Representative computer readable signals, whether modulated using a carrier or not, are signals that a computer system hosting or running the computer program may be configured to access, including signals downloaded through the Internet or other networks. Concrete examples of the foregoing include distribution of the programs on a CD ROM or via Internet download. In a sense, the Internet itself, as an abstract entity, is a computer readable medium. The same is true of computer networks in general

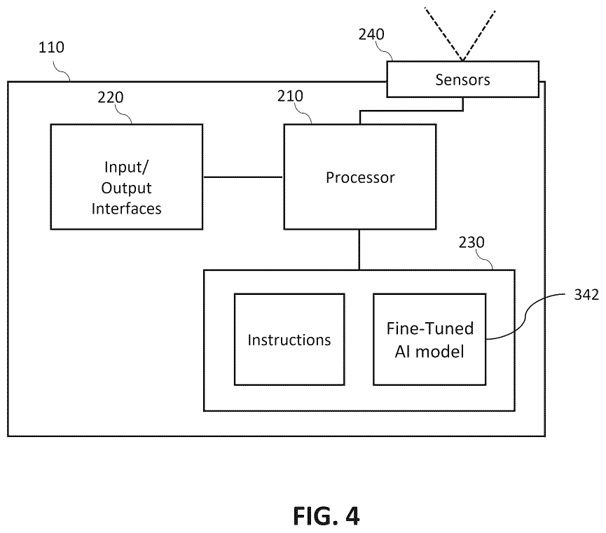

FIG. 4 is an illustrative representation of the MR device 110. In this implementation, the MR device 110 is self-contained in that it is a single head-mounted device that incorporates hardware that is capable of locally carrying out the operations of real-time recognition and tracking of the patient's face, and superimposing visual data on the patient's. It is appreciated, however, that in other implementations, some of the operations can be offloaded from the device, and for example be carried out remotely, such as on a server or other separate device.

In the present implementation, the MR device 110 includes a processor. The processor 210 may include a general-purpose processor, such as a central processing unit (CPU) or a processor dedicated to a specific purpose, such as a digital signal processor (DSP). In some implementations, the processor 210 may also rely on an accelerator dedicated to certain given tasks, such as executing specific operations described herein. In some implementations, the processor 210 or the accelerator may be implemented as one or more field programmable gate arrays (FPGAs). Moreover, explicit use of the term “processor”, should not be construed to refer exclusively to hardware capable of executing software, and may implicitly include, without limitation, application-specific integrated circuit (ASIC), read-only memory (ROM) for storing software, RAM, and non-volatile storage. Other hardware, conventional and/or custom, may also be included.

The processor 210 is operatively connected to memory 230 and to input/output interfaces 220. The memory 230 includes persistent storage 234 for storing the fine-tuned AI model 342 and for storing instructions executable by the processor 210 to cause the MR device 120 to perform the operations discussed above.

The input/output interfaces 220 may provide networking capabilities such as wired or wireless access. As an example, the input/output interface 220 may comprise a networking interface such as, but not limited to, one or more network ports, one or more network sockets, one or more network interface controllers and the like. Multiple examples of how the networking interface may be implemented will become apparent to the person skilled in the art of the present technology. For example, but without being limitative, the networking interface may implement specific physical layer and data link layer standard such as Ethernet, Fibre Channel, Wi-Fi. The specific physical layer and the data link layer may provide a base for a full network protocol stack, allowing communication among small groups of computers on the same local area network (LAN) and large-scale network communications through routable protocols, such as Internet Protocol (IP). The networking interface can allow the MR device 110 to communicate with other computing devices, for example to communicate with the AI model generating system 301 to receive the fined-tuned AI model 342 therefrom.

The input/output interfaces 220 may also include a display. The display may be a screen and/or projection device capable of presenting an augmented view 120 to an operator. For example, in the present implementation, the display is a head mounted display that is worn by the operator. In other implementations, the display may be remotely communicatively connected to the MR device 110 via a wired or a wireless connection (not shown), so that the augmented view can be presented at a location different from the location of the MR device 110. In this situation, the display may be operationally coupled to, but housed separately from, other functional units and systems in the MR device 110.

The MR device may also include a plurality of sensors 240. The sensors 240 can include one or more optical sensors mounted on a front surface of the MR device 110. For example, the optical sensors may be configured to capture Red-Green-Blue (RGB) images. The optical sensors may comprise image sensors such as, but not limited to, Charge-Coupled Device (CCD) or Complementary Metal Oxide Semiconductor (CMOS) sensors, digital cameras, depth cameras, etc. The optical sensors may convert an optical image into an electronic or digital image and may send captured images to the processor 210. In the same or other implementations, the optical sensors may be a single-lens camera providing RGB pictures. In some implementations, the optical sensors include depth sensors to acquire RGB-Depth (RGBD) pictures. Broadly speaking, any device suitable for capturing an image of the patient and/or the physical space around the patient may be used as the optical sensors.

In some implementations, the sensors 240 may include an Inertial Sensing Unit (ISU) configured to be used in part by the processor 210 to determine a position and orientation of the optical sensors and/or of the MR device 110. Therefore, such sensors can allow determining a set of coordinates describing the location of the MR device 110, for example in a coordinate system based on the output of the ISU. The ISU may, for example, include 3-axis accelerometer(s), 3-axis gyroscope(s), and/or magnetometer(s) and may provide velocity, orientation, and/or other position related information to the processor 210.

In the foregoing specification, specific embodiments have been described. However, one of ordinary skill in the art appreciates that various modifications and changes can be made without departing from the scope of the present teachings. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims.

1. A method for superimposing visual data relative to a patient using a mixed reality device, the method comprising:
accessing a plurality of 3D scans of the patient's face;
extracting patient-specific facial features from the plurality of 3D scans;
fine-tuning a pre-trained generic 3D facial recognition AI model using the patient-specific facial features to more accurately recognize the patient's face;
performing real-time recognition and tracking of the patient's face on the mixed reality device using the fine-tuned model; and
superimposing the visual data relative to the patient's tracked face using the mixed reality device.

2. The method of claim 1, wherein the plurality of 3D scans comprise a plurality of 3D face scans of the patient from different angles and expressions.

3. The method of claim 1, wherein fine-tuning the generic 3D facial recognition AI model comprises adjusting weights of layers of the generic 3D facial recognition AI model based on the patient-specific facial features.

4. The method of claim 1, wherein the visual data comprises a 3D model associated with a facial structure of the patient, and superimposing the visual data comprises determining a real-time position of the facial structure in 3D space according to the patient's tracked face, and projecting the 3D model in alignment with the determined position of the facial structure.

5. The method of claim 4, wherein the facial structure comprises a dental structure.

6. The method of claim 4, wherein the 3D model comprises at least one of: a 3D model of a denture, a 3D model of a virtual surgical or restorative planning, a virtual 3D smile design, a 3D model of a virtual orthodontic or orthognathic planning, a 3D model of a dental implant, a 3D model of operations of a periodontal, orthognathic or endodontic surgical guide, a 3D representation of a treatment plan, a 3D model of a patient's teeth, oral, maxillofacial or other anatomical facial structures.

7. A computer-implemented method for training a personalized artificial intelligence (AI) model for real-time recognition and tracking of a patient's face, the method comprising:
accessing three-dimensional (3D) features of the patient's face;
partitioning the 3D features into a training set of 3D features and a testing set of 3D features;
accessing a pre-trained generic AI model of a generic face, the generic AI model being configured to generate visual data about the generic face based on 3D features thereof;
fine-tuning the pre-trained generic AI model based on the 3D features of the training set; and
evaluating a performance of the fine-tuned AI model based on the testing set.

8. A system for superimposing visual data relative to a patient, the system comprising:
a controller;
a memory communicably connected to the controller and storing a plurality of executable instructions;
an imaging device communicably connected to the controller and configured to capture 3D scans of a patient's face; and
a mixed reality device communicably connected to the controller,
wherein:
the controller is configured to cause the system to, upon executing the plurality of executable instructions:
extract patient-specific facial features from the plurality of 3D scans;
fine-tune a pre-trained generic 3D facial recognition AI model using the patient-specific facial features to generate visual data relative to the patient's face;
perform real-time recognition and tracking of the patient's face on the mixed reality device using the fine-tuned AI model; and
superimpose the visual data on the patient's tracked face using the mixed reality device.

9. The system of claim 8, wherein the visual data is superimposed on the patient's tracked face during an execution of a real time live dental, oral or maxillofacial procedure, endodontic, orthodontic and periodontic procedures.

10. The system of claim 9, wherein the procedure is an endodontic, orthodontic or periodontic procedure, the procedure being a surgical or a restorative procedure.

11. The system of claim 8, wherein the plurality of 3D scans comprise a plurality of 3D face scans of the patient from different angles and expressions.

12. The system of claim 8, wherein fine-tuning the generic 3D facial recognition AI model comprises adjusting weights of layers of the generic 3D facial recognition AI model based on the patient-specific facial features.

13. The system of claim 8, wherein the visual data comprises a 3D model associated with a facial structure of the patient, and superimposing the visual data comprises determining a real-time position of the facial structure in 3D space according to the patient's tracked face, and projecting the 3D model in alignment with the determined position of the facial structure.

14. The system of claim 13, wherein the facial structure comprises a dental structure.

15. The system of claim 13, wherein the 3D model comprises at least one of: a 3D model of a denture, a 3D model of a virtual surgical or restorative planning, a virtual 3D smile design, a 3D model of a virtual orthodontic or orthognathic planning, a 3D model of a dental implant, a 3D model of operations of a periodontal, orthognathic or endodontic surgical guide, a 3D representation of a treatment plan, a 3D model of a patient's teeth, oral, maxillofacial or other anatomical facial structures.
